(19) United States
(12) Patent Application Publication
(10) Pub. No.: US 2019/0070136 A1
(43) Pub. Date: Mar. 7, 2019

(54) PARENTERAL COMPOSITIONS OF CARMUSTINE

(71) Applicant: ASPIRO PHARMA LIMITED, HYDERABAD (IN)

(72) Inventors: BANDI PARTHASARADHI REDDY, Medak (IN); PODILE KHADGAPATHI, Medak (IN); BANDARI SREEDHAR, Medak (IN)

(21) Appl. No.: 16/120,608

(22) Filed: Sep. 4, 2018

(30) Foreign Application Priority Data
Sep. 4, 2017 (IN) 201741031264

(51) Int. Cl. A61K 31/175; A61K 9/00; A61K 47/10; A61K 47/18; A61K 47/22

(52) U.S. Cl. CPC A61K 31/175; A61K 9/0053; A61K 47/22; A61K 47/18; A61K 47/10

(57) ABSTRACT

Described herein are parenteral compositions of carmustine. More particularly, described herein are ready to use parenteral compositions of carmustine in the form of solutions.

### CROSS-REFERENCE TO RELATED APPLICATIONS

This application claims priority to Indian provisional Application IN201741031264 filed on Sep. 4, 2017, which is incorporated herein by reference in its entirety.

### FIELD OF THE DISCLOSURE

The present invention relates to parenteral compositions of carmustine and process for the preparation thereof.

### BACKGROUND

Carmustine is a β-chloro-nitrosourea compound useful in chemotherapy of certain neoplastic diseases. It has the chemical name, 1,3-bis (2-chloroethyl)-1-nitrosourea, with a molecular weight of 214.06 and its empirical formula is C₅H₉Cl₂N₃O₂, with the following structure:

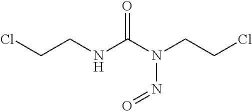 [SMILES: O=NN(CCCl)C(=O)NCCCl]

It is very soluble in alcohol, such as tertiary butanol, dichloromethane, and ether, and slightly soluble in water with a solubility of 4 mg/mL. Carmustine is readily hydrolyzed in water at pH>6. Carmustine has a Log P value of 1.53. Its anti-neoplastic activity is mainly due to its effect on DNA, RNA, mitochondrial glutathion reductase and Cytochrome P450 enzymes.

Carmustine is commercially available as a sterile lyophilized powder for injection under the tradename BiCNU®, indicated for the treatment of brain tumors, multiple myeloma, Hodgkin's disease, and non-Hodgkin lymphoma (NHL).

BiCNU® (carmustine for injection) is available in single dose vials as lyophilized powder containing 100 mg of carmustine, co-packaged with ethanol as a sterile diluent. The lyophilized carmustine appears as pale yellow dry flakes or a dry congealed mass. Prior to injection, the lyophilized carmustine is reconstituted with ethanol and the solution is further diluted with water. The reconstitution results in a clear, colorless to yellowish solution which is further diluted with 5% dextrose injection, USP or sodium chloride Injection, USP.

Carmustine has poor stability in aqueous medium and is slightly soluble in water and thus forms a cloudy solution or suspension resulting in reduced drug efficiency in vivo.

The powder formulation of carmustine possesses good chemical stability. However, reconstitution of the powder is an inconvenient and time consuming process.

Thus, there is a need to develop improved, robust, non-powder formulations of carmustine prepared by a simple process without using the lyophilization technique.

### SUMMARY

The present invention relates to liquid parenteral compositions of carmustine.

One embodiment relates to a “ready-to-use” non-aqueous liquid formulation comprising carmustine for parenteral administration.

Another embodiment relates to a “ready-to-dilute” non-aqueous liquid formulation comprising carmustine for parenteral administration.

Another embodiment relates to ready to use parenteral composition comprising carmustine or its pharmaceutically acceptable salt, one or more solvents selected from propylene glycol, polyethylene glycol, dimethylacetamide, N-methyl pyrrolidone, diethylene glycol monoethyl ether, monothioglycerol, dehydrated alcohol, and mixtures thereof, optionally other pharmaceutically acceptable excipients.

Another embodiment relates to parenteral compositions comprising carmustine, N, N-dimethylacetamide and polyethylene glycol.

Another embodiment relates to parenteral compositions comprising carmustine, N, N-dimethylacetamide and propylene glycol.

A further embodiment relates to parenteral compositions comprising carmustine and diethylene glycol monoethyl ether (Transcutol®).

Yet another embodiment relates to parenteral compositions comprising carmustine and N-methyl pyrrolidone.

Yet another embodiment relates to parenteral compositions comprising carmustine, N,N dimethyl acetamide, propylene glycol, monothioglycerol, and dehydrated alcohol.

Yet another embodiment provides a pharmaceutical composition comprising 25 mg/mL or 100 mg/mL of Carmustine, one or more solvents selected from propylene glycol, dimethylacetamide, N-methyl pyrrolidone, polyethylene glycol, monothioglycerol, dehydrated alcohol and mixtures thereof, having not more than 1% of Impurity A.

Another embodiment relates to use of carmustine compositions of the present disclosure for the treatment of certain neoplastic diseases, specifically, brain tumors, multiple myeloma, Hodgkin's disease, or non-Hodgkin lymphoma.

### DETAILED DESCRIPTION

The present invention relates to liquid parenteral compositions of carmustine. More particularly, the present invention relates to ready to use parenteral compositions of carmustine in the form of solutions.

The term “active ingredient” or “active agent” or “drug” are used interchangeably, and are defined to mean active drug (e.g. carmustine) that induces a desired pharmacological or physiological effect.

The term “pharmaceutically acceptable” as used herein means that which is useful in preparing a pharmaceutical composition that is generally safe and non-toxic.

The term “excipients” herein means a component of a pharmaceutical product that is not an active ingredient. The excipients that are useful in preparing a pharmaceutical composition are generally safe and non-toxic.

The term “parenteral” as used herein means administration through intravenous, intramuscular, subcutaneous, intracutaneous, intra-articular, intrathecal routes.

As used in the specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “solvent” refers to an ingredient used for dissolving another ingredient. Exemplary solvents include, but are not limited to, propylene glycol, dimethyl acetamide (N, N-dimethylacetamide/DMA), N-methyl pyrrolidone, polyethylene glycol of various molecular weights, polyethylene glycol 400 (PEG-400), polyethylene glycol 800 (PEG-800), diethylene glycol monoethyl ether (Transcutol®), dimethyl sulfoxide (DMSO), ethanol, methanol, tertiary-butyl alcohol, isopropyl alcohol, methylene chloride, ethyl acetate, acetone, monothioglycerol, dehydrated alcohol, and combinations thereof. Preferred solvents include propylene glycol, polyethylene glycol, N-methyl pyrrolidone, dimethyl acetamide, monothioglycerol, dehydrated alcohol and diethylene glycol monoethyl ether.

Diethylene glycol monoethyl ether is a highly purified solvent for poorly water soluble active pharmaceutical ingredients, marketed by Gattefosse under the brand name Transcutol®.

One embodiment of the present invention relates to ready to use parenteral composition comprising carmustine or its pharmaceutically acceptable salt, propylene glycol, N, N-dimethyl acetamide, N-methyl pyrrolidone, polyethylene glycol, diethylene glycol monoethyl ether, monothioglycerol, dehydrated alcohol, and optionally one or more surfactants.

The composition according comprises about 10 mg to about 100 mg of carmustine for each ml of solvent, specifically about 25 mg of carmustine for each ml of solvent or about 100 mg of carmustine for each ml of solvent.

In an aspect, a pharmaceutical composition comprises 25 mg/mL or 100 mg/mL of Carmustine, one or more solvents selected from propylene glycol, dimethylacetamide, N-methyl pyrrolidone, polyethylene glycol, monothioglycerol, dehydrated alcohol, and mixtures thereof, having not more than 1% of Impurity A.

Chemical Impurity A is 1,3-Bis(2-chloroethyl)urea.

Another embodiment of the present invention relates to a process for the preparation of pharmaceutical compositions of carmustine, the process comprising:

(a) adding a weighed quantity of carmustine to a non-aqueous solvent in a vessel with continuous stirring until the carmustine is dissolved completely,

(b) filtering the solution of step (a), and filling the filtered solution into vials, and

(c) stoppering and sealing the vials of step (b).

Another embodiment of the present invention relates to a process for the preparation of pharmaceutical compositions of carmustine, the process comprising:

(a) adding a weighed quantity of carmustine to N,N dimethyl acetamide and propylene glycol in a vessel with continuous stirring until the carmustine is dissolved completely,

(b) filtering the solution of step (a) and filling the filtered solution into vials, and

(c) stoppering and sealing the vials of step (b).

Another embodiment of the present invention relates to a process for the preparation of pharmaceutical compositions of carmustine, the process comprising:

(a) adding a weighed quantity of carmustine to N,N dimethyl acetamide and polyethylene glycol in a vessel with continuous stirring until the carmustine is dissolved completely,

(b) filtering the solution of step (a), and filling the filtered solution into vials, and

(c) stoppering and sealing the vials of step (b).

Another embodiment of the present invention relates to a process for the preparation of pharmaceutical compositions of carmustine, the process comprising:

(a) adding a weighed quantity of carmustine to diethylene glycol monoethyl ether, and optionally other solvents in a vessel with continuous stirring until the carmustine is dissolved completely,

(b) filtering the solution of step (a), and filling the filtered solution into vials, and

(c) stoppering and sealing the vials of step (b).

Another embodiment of the present invention relates to a process for the preparation of pharmaceutical compositions of carmustine, the process comprising:

(a) adding a weighed quantity of carmustine to N-methyl pyrrolidone in a vessel with continuous stirring until the carmustine is dissolved completely,

(b) filtering the solution of step (a), and filling the filtered solution into vials, and

(c) stoppering and sealing the vials of step (b).

Another embodiment of the present invention relates to process for the preparation of pharmaceutical compositions of carmustine, the process comprising:

(a) addition of weighed quantity of carmustine, N, N dimethyl acetamide, propylene glycol, monothioglycerol, and dehydrated alcohol in a vessel with continuous stirring until the carmustine is dissolved completely,

(b) filtering the solution of step (a), and filling the filtered solution into vials, and

(c) stoppering and sealing the vials of step (b).

The compositions optionally further comprise one or more other pharmaceutically acceptable excipients selected from a bulking agent, a solubilizer, a surface active agents, a buffer, a pH adjustment aid, a chelating agent, an antioxidant, an antibacterial preservative or combinations thereof.

Bulking agents include but not limited to mannitol, lactose, sucrose, sodium chloride, trehalose, dextrose, starch, hydroxyethyl starch, cellulose, cyclodextrins, glycine, and mixtures thereof.

Solubilizers can be surface active agents, co-solvents, complexing agents and combinations thereof.

Surface active agents are hydrophilic in nature and include but are not limited to sorbitan fatty acid esters, polysorbates, poloxamers, oleoyl and linoleoyl polyoxylglycerides (such as Labrafil®), caprylocaproylpolyoxylglycerides (such as Labrasol), medium-chain triglycerides (such as Labrafac® lipophile), propylene glycol dicaprylocaprate (such as Labrafac® PG), and mixtures thereof.

Buffers include an acid or a base and a conjugate base or acid, respectively. Exemplary buffers include mixtures of weak acids and alkali metal salts (e.g., sodium, potassium) of the weak acids, such as acetate, citrate, tartarate, phosphate, benzoate and bicarbonate buffers, and combinations thereof.

pH adjustment aids include but are not limited to tartaric acid, citric acid, malic acid, sodium chloride, potassium chloride, sodium hydroxide, potassium hydroxide, sodium carbonate, meglumine, and combinations thereof.

Chelating agents include but are not limited to ethylenetetraamineacetic acid, ethylenediaminetetraacetic acid (EDTA), and salts, derivatives and combinations thereof.

Compositions of the present invention can be directly administered or can be diluted using sodium chloride, e.g., 0.9% sodium chloride injection, USP, or dextrose, e.g., 2.5% or 5% dextrose/0.45% sodium chloride injection, USP before parenteral administration.

Liquid dosage forms according to the present invention may be “ready-to-use” or “ready to dilute” formulations.

The term “ready to use” composition as used herein refers to the composition which avoids reconstitution and may require dilution with a suitable diluent before administration to the patient.

The term “ready to dilute” compositions according to the present invention requires a single dilution before administering to a patient.

The composition of the present invention is useful for the treatment of certain neoplastic diseases, brain tumors, multiple myeloma, Hodgkin's disease, or non-Hodgkin lymphoma.

### EXAMPLES

The following examples further describe and demonstrate particular embodiments within the scope of the present invention. The examples are given solely for illustration and are not to be construed as limitations as many variations are possible without departing from spirit and scope of the invention. It is obvious to those skilled in the art to find out the composition for other dosage forms and substitute the equivalent excipients as described in this specification or with the one known to the industry.

### Example 1

Parenteral compositions of Carmustine

| Ingredients | Qty/mL |
|---|---|
| Carmustine | 100 mg |
| Propylene glycol | 320 mg |
| N,N Dimethylacetamide | 580 mg |

Brief Manufacturing Process:

1. A measured quantity of N, N-dimethylacetamide and propylene glycol were transferred to a clean and dried SS316 vessel,

2. a weighed quantity of carmustine was dissolved in step (1) with continuous stirring until a clear solution was obtained,

3. the final solution of step (2) was filtered through a sterile filter, and

4. the filtered solution of step (3) was filled into the 5 mL/20 mm neck Type-I glass vials with the fill volume of NLT 1 mL, the vials were stoppered and sealed and stored at 2° C.-8° C.

### Example 2

| Ingredients | Qty/mL |
|---|---|
| Carmustine | 100 mg |
| Polyethylene glycol | 320 mg |
| N,N Dimethylacetamide | 580 mg |

Brief Manufacturing Process:

1. A measured quantity of polyethylene glycol and N, N-dimethylacetamide were transferred to a clean and dried SS316 vessel,

2. A weighed quantity of carmustine was dissolved in step (1) with continuous stirring until a clear solution was obtained,

3. the final solution of step (2) was filtered through a sterile filter, and

4. the filtered solution of step (3) was filled into the 5 mL/20 mm neck Type-I glass vials with the fill volume of NLT 1 mL, the vials were stoppered and sealed and stored at 2° C.-8° C.

### Example 3

| Ingredients | Qty/mL | |
|---|---|---|
| Carmustine | 100 | mg |
| diethylene glycol monoethyl ether | Q.s to 1 | ml |

Brief Manufacturing Process:

1. A measured quantity of diethylene glycol monoethyl ether was transferred to in a clean and dried SS316 vessel,

2. A weighed quantity of carmustine was dissolved in step (1) with continuous stirring until a clear solution was obtained,

3. the final solution of step (2) was filtered through a sterile filter, and

4. the filtered solution of step (3) was filled into the 5 mL/20 mm neck Type-I glass vials with the fill volume of NLT 1 mL, the vials were stoppered and sealed and stored at 2° C.-8° C.

### Example 4

| Ingredients | Qty/mL | |
|---|---|---|
| Carmustine | 100 | mg |
| N-methyl Pyrrolidine | Q.s to 1 | ml |

Brief Manufacturing Process:

1. A measured quantity of N-methyl pyrrolidone was transferred to a clean and dried SS316 vessel,

2. A weighed quantity of carmustine was dissolved in step (1) with continuous stirring until a clear solution was obtained,

3. the final solution of step (2) was filtered through a sterile filter, and

4. the filtered solution of step (3) was filled into the 5 mL/20 mm neck Type-I glass vials with the fill volume of NLT 1 mL, the vials were stoppered and sealed and stored at 2° C.-8° C.

### Example 5

| Ingredients | Qty/mL | |
|---|---|---|
| Carmustine | 100 | mg |
| N,N Dimethyl acetamide | 300 | mg |
| Propylene glycol | 1 | mg |
| Monothioglycerol | 1 | mg |
| Dehydrated alcohol | Q.s to 1 | ml |

Brief Manufacturing Process:

1. A measured quantity of N,N Dimethyl acetamide, propylene glycol, monothioglycerol and dehydrated alcohol were transferred to a clean and dried SS316 vessel,

2. A weighed quantity of carmustine was dissolved in step (1) with continuous stirring until a clear solution was obtained,

3. the final solution of step (2) was filtered through a sterile filter, and

4. the filtered solution of step (3) was filled into the 5 mL/20 mm neck Type-I glass vials with the fill volume of NLT 1 mL, the vials were stoppered and sealed and stored at 2° C.-8° C.

### Stability Studies:

The compositions prepared according to example 1-5 were stored at 2° C.-8° C. and tested for assay % and impurities. The results are as follows:

| | Description | | |
|---|---|---|---|
| | (clear yellow | Assay (%) | Impurity A |
| Contents | colour solution) | (90-110%) | (NMT 1.0%) |
| Example 1 | Complies | 105.5% | Not detected |
| Example 2 | Complies | 102.7% | Not detected |
| Example 3 | Complies | 103.2 | Not detected |
| Example 4 | Complies | 105.0% | 0.1% |
| Example 5 | Complies | 98.1% | 0.05% |

Above data reveals that liquid formulations of carmustine injection were found to be compatible with the excipients used and analytical parameters were found to be with in the specified limits.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

We claim:

1. A ready to use liquid parenteral composition comprising:
(i) carmustine or a pharmaceutically acceptable salt thereof, and
(ii) one or more solvents selected from propylene glycol, dimethylacetamide, N-methyl pyrrolidone, polyethylene glycol, monothioglycerol, dehydrated alcohol, and mixtures thereof.

2. The ready to use parenteral composition according to claim 1, wherein the composition is in the form of a solution.

3. The ready to use parenteral composition according to claim 1, comprising about 25 mg/ml to about 100 mg/ml of carmustine.

4. The ready to use parenteral composition according to claim 1, wherein the composition further comprises one or more pharmaceutically acceptable excipients selected from a bulking agent, a solubilizer, a buffer, a pH adjustment aid, a chelating agent, an antioxidant, an antibacterial preservative, and combinations thereof.

5. A parenteral composition comprising 25 mg/mL or 100 mg/mL of carmustine; one or more solvents selected from propylene glycol, dimethylacetamide, N-methyl pyrrolidone, polyethylene glycol, monothioglycerol, dehydrated alcohol, and mixtures thereof; the composition having not more than 1% of Impurity A.

6. A method of making a ready to use parenteral solution composition according to claim 1, comprising:
(a) adding a weighed quantity of carmustine to one or more solvents selected from N-methyl pyrrolidone, N, N dimethylacetamide, propyleneglycol, polyethylene glycol, monothioglycerol, dehydrated alcohol, and mixtures thereof and stirring continuously until complete dissolution of the carmustine,
(b) filtering the solution and filling the filtered solution into vials, and
(c) stoppering and sealing the vials.

7. A method of treating a patient, the method comprising diluting the composition of claim 1 before administering the composition to the patient.

8. The method of claim 7, wherein the composition is diluted with sodium chloride injection or dextrose.

9. The method of treating a neoplastic diseases, wherein at least one disease selected from brain tumors, multiple myeloma, Hodgkin's disease, and non-Hodgkin lymphoma in a patient in need thereof, comprising administering to the patient the composition of claim 1.
